(19) United States
(12) Patent Application Publication
(10) Pub. No.: US 2022/0189607 A1
(43) Pub. Date: Jun. 16, 2022

(54) METHODS AND APPARATUS FOR RECOMMENDING TAILORED WELLNESS ACTIVITIES BASED UPON NON-WELLNESS-RELATED DATA

(71) Applicant: STATE FARM MUTUAL AUTOMOBILE INSURANCE COMPANY, Bloomington, IL (US)

(72) Inventors: Edward W. Breitweiser, Bloomington, IL (US); Jennifer L. Crawford, Normal, IL (US); Brian Steigerwald, Bloomington, IL (US); Danielle Malan, Downs, IL (US)

(21) Appl. No.: 17/550,478

(22) Filed: Dec. 14, 2021

Related U.S. Application Data
(60) Provisional application No. 63/125,668, filed on Dec. 15, 2020.

(51) Int. Cl. G16H 20/30; G06Q 30/02

(52) U.S. Cl. CPC G16H 20/30; G06N 20/00; G06Q 30/0224

(57) ABSTRACT

Methods and apparatus for recommending tailored wellness activities based upon non-wellness-related data are disclosed. In an embodiment, a computer-implemented method for recommending wellness activities based upon non-wellness-related data includes accessing non-wellness-related data for a person from a datastore. The data is processed to determine a propensity score, the propensity score representing a likelihood that the person would perform a wellness activity. When the propensity score satisfies a condition, a wellness activity related to an aspect of the data is identified, and information regarding the wellness activity to the person is communicated via a network interface.

### RELATED APPLICATION

This application claims the priority benefit of U.S. Provisional Patent Application No. 63/125,668, entitled “Methods And Apparatus For Recommending Tailored Wellness Activities Based Upon Non-Wellness-Related Data,” and filed on Dec. 15, 2020. U.S. Provisional Patent Application No. 63/125,668 is hereby incorporated herein by reference in its entirety.

### FIELD OF THE DISCLOSURE

This disclosure relates generally to lifestyle management systems and, more particularly, to methods and apparatus for recommending tailored wellness activities based upon non-wellness-related data.

### BACKGROUND

Many people are interested in wellness or well-being activities, such as walking, bicycling, etc. Accordingly, entities such as insurers and employers, who are interested in promoting the well-being of their clients and employees, may often suggest wellness activities. For such suggestions to be effective, however, they must be made to individuals who have a propensity to consider acting on such suggestions. Effectiveness may be further enhanced when such suggestions are made at an appropriate time. Current techniques for suggesting well-being activities may have limited effectiveness because they are presented as generic recommendations to groups of individuals who may or may not be predisposed to act on such suggestions. Conventional techniques may have other drawbacks as well.

### BRIEF SUMMARY

The present embodiments relate to, inter alia, mining non-wellness-related data to obtain information that may be useful in identifying or suggesting wellness activities that are tailored to a particular person. The data may be processed to determine a likelihood that the person would perform a wellness activity. When, for example, the likelihood exceeds a threshold, an aspect of the data may be used to identify a wellness activity, and information regarding the wellness activity may be communicated to the person. For example, when financial and/or insurance information indicates a person bought a bicycle, the present embodiments may suggest a wellness activity tailored to involve a bicycle.

In one aspect, a computer-implemented method for recommending wellness activities based upon non-wellness-related data may include accessing non-wellness-related data for a person from a datastore. The data may be processed to determine a propensity score, the propensity score representing a likelihood that the person would perform a wellness activity. When the propensity score satisfies a condition, a wellness activity related to an aspect of the data may be identified, and information regarding the wellness activity may be communicated to the person. The method may include additional, less, or alternate functionality or actions, including those discussed elsewhere herein.

In another aspect, a computer system for recommending wellness activities based upon non-wellness-related data may include a data miner configured to access non-wellness-related data for a person from a datastore. The data may be processed with a propensity model configured to determine a propensity score, wherein the propensity score represents a likelihood that the person would perform a wellness activity. The system may include an activity identifier configured to, when the propensity score satisfies a condition, identify a wellness activity related to an aspect of the data. A network interface of the system may be configured to communicate information regarding the wellness activity to the person. The system may include additional, less, or alternate functionality, including that discussed elsewhere herein.

### BRIEF DESCRIPTION OF THE DRAWINGS

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

The figures described below depict various aspects of the applications, methods, and systems disclosed herein. It should be understood that each figure depicts an embodiment of a particular aspect of the disclosed applications, systems and methods, and that each of the figures is intended to accord with one or more possible embodiments thereof. Furthermore, wherever possible, the following description refers to the reference numerals included in the following figures, in which features depicted in multiple figures are designated with consistent reference numerals.

FIG. 1 illustrates an exemplary wellness activity recommendation system, in accordance with disclosed embodiments.

FIG. 2 is a flowchart representative of an exemplary computer-implemented method, hardware logic or machine-readable instructions for implementing the exemplary wellness activity server of FIG. 1, in accordance with disclosed embodiments.

FIG. 3 is a flowchart representative of an exemplary computer-implemented method, hardware logic or machine-readable instructions for implementing the exemplary monitor and incentive systems of FIG. 1, in accordance with disclosed embodiments.

FIG. 4 is a block diagram of an exemplary computing system to implement the various disclosed user interfaces, methods, functions, etc., for recommending tailored wellness activities.

The figures depict preferred embodiments for purposes of illustration only. One skilled in the art will readily recognize from the following discussion that alternative embodiments of the systems and methods illustrated herein may be employed without departing from the principles of the invention described herein.

### DETAILED DESCRIPTION

Often wellness activity suggestions are generic, and the same suggestions are made to many people. Additionally, existing systems tailor wellness activities based on a person's past responses to suggested wellness activities or the completion of suggested wellness activities. Such limitations may fail to keep a person interested and engaged in carrying out wellness activities. Accordingly, to reduce or eliminate some or all of these or other problems, disclosed methods and apparatus may recommend tailored wellness activities based upon alternative, non-wellness-related sources of data, such as insurance-related information, financial-related information, property record information, social media information, etc.

Accordingly, the disclosed methods and apparatus may mine such non-wellness-related data to obtain information that does not exist in wellness-related data but may be used to identify and suggest wellness activities that are tailored to a particular person. For example, when financial and/or insurance information indicates a person bought a bicycle, disclosed methods and apparatus may suggest a wellness activity involving a bicycle. Also, when home and/or work-related information indicates a person lives near where they work, disclosed methods and apparatus may suggest a wellness activity involving walking or biking to work. Further, when home and/or work-related information indicates a person lives and/or works near a park, disclosed methods and apparatus may suggest a wellness activity involving the park. As yet another example, when home and/or traffic information indicates a person lives near roads that are safe for biking, disclosed methods and apparatus may not suggest a wellness activity near home that involves a bike.

In one aspect, a computer-implemented method for recommending wellness activities based upon non-wellness-related data may include accessing non-wellness-related data for a person from a datastore. The data may be processed to determine a propensity score, the propensity score representing a likelihood that the person would perform a wellness activity. When the propensity score satisfies a condition, a wellness activity related to an aspect of the data may be identified, and information regarding the wellness activity may be communicated to the person. The method may include additional, less, or alternate functionality or actions, including those discussed elsewhere herein.

For instance, in one or more variations of the current embodiment, the computer-implemented method may further include updating the propensity model based upon feedback regarding the wellness activity. In one or more variations of the current embodiment, the feedback may include an indication from the person of at least one of no interest in the wellness activity, potential interest in the wellness activity, or completion of the wellness activity; and/or the feedback may be generated by a personal computing device that automatically tracks completion of wellness activities. Additionally or alternatively, the computer-implemented method may further include collecting, using one or more processors, feedback regarding the wellness activity, and/or awarding, using one or more processors, an incentive based upon the feedback.

In one or more variations of the current embodiment, the propensity model may include a machine learning algorithm updated for the person based upon feedback regarding the wellness activity. The computer-implemented method may further include modifying an aspect of the wellness activity based upon additional non-wellness-related data for the person from the datastore or another datastore.

In one or more variations of the current embodiment, the datastore may store at least one of insurance-related information, financial-related information, property record information, or social media information. The data may represent ownership of a piece of equipment, and the identified wellness activity includes a use of the piece of equipment; and/or the data may represent opening of a new wellness activity area, and the wellness activity may include use of the new wellness activity area.

In another aspect, a computer system for recommending wellness activities based upon non-wellness-related data may include a data miner configured to access non-wellness-related data for a person from a datastore. The data may be processed with a propensity model configured to determine a propensity score, wherein the propensity score represents a likelihood that the person would perform a wellness activity. The system may include an activity identifier configured to, when the propensity score satisfies a condition, identify a wellness activity related to an aspect of the data. A network interface of the system may be configured to communicate information regarding the wellness activity to the person. The system may include additional, less, or alternate functionality, including that discussed elsewhere herein.

For instance, in one or more variations of the current embodiment, the system may further include a monitor system configured to collect feedback regarding the wellness activity, wherein the propensity model may be configured to update based upon the feedback. The system may further include a monitor system configured to collect feedback regarding the wellness activity, and an incentive system configured to award an incentive based upon the feedback.

In one or more variations of the current embodiment, the activity identifier may be configured to modify an aspect of the wellness activity based upon additional non-wellness-related data for the person from the datastore or another datastore. The datastore may store at least one of insurance-related information, financial-related information, property record information or social media information.

In yet another embodiment, a non-transitory computer-readable storage medium may store instructions that, when executed by one or more processors, cause a system to access non-wellness-related data for a person from a datastore, process the data to determine a propensity score, wherein the propensity score represents a likelihood that the person would perform a wellness activity, when the propensity score satisfies a condition, identify a wellness activity related to an aspect of the data, and communicate information regarding the wellness activity to the person. The instructions may direct additional, less, or alternate functionality, including that discussed elsewhere herein.

For instance, in one or more variations of the current embodiment, the instructions, when executed by the one or more processors, may cause the system to collect feedback regarding the wellness activity, and/or update a model used to process the propensity score based upon the feedback. The instructions, when executed by the one or more processors, may cause the system to collect feedback regarding the wellness activity, and award an incentive based upon the feedback.

In one or more variations of the current embodiment, the instructions, when executed by the one or more processors, may cause the system to modify an aspect of the wellness activity based upon additional non-wellness-related data for the person from the datastore or another datastore. The datastore may store at least one of insurance-related information, financial-related information, property record information or social media information.

Reference will now be made in detail to non-limiting examples, some of which are illustrated in the accompanying drawings.

### Exemplary Wellness Activity Recommendation System

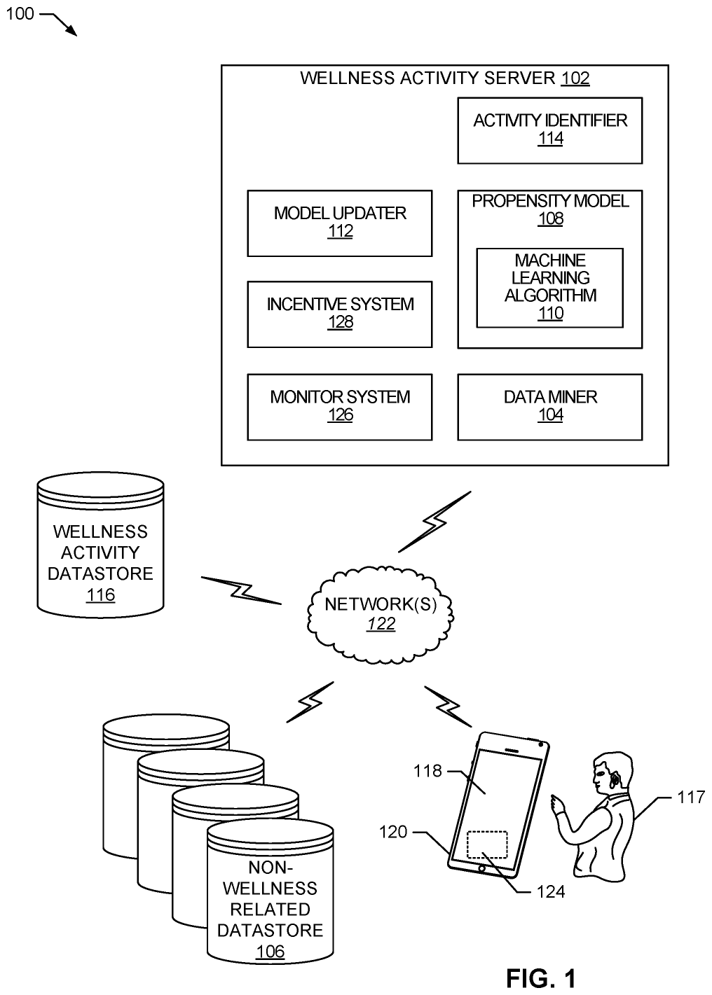

FIG. 1 illustrates an exemplary wellness activity recommendation system 100, in accordance with disclosed embodiments. To identify and/or recommend wellness activities, the exemplary wellness activity recommendation system 100 may include an exemplary wellness activity server 102.

To obtain data and/or information from which a wellness activity may potentially be identified and/or modified, the wellness activity server 102 may include an exemplary data miner 104. The data miner 104 may mine, obtain, access, collect, etc. information and/or data from any number and/or type(s) of datastores, data sources, databases, etc. (one of which is designated at reference numeral 106) storing non-wellness related data. For example, the data miner 104 may access one or more of the datastores 106 to determine if a person owns a bicycle or other fitness related equipment, to determine if a person lives or works near a park, to determine if a person lives close enough to work to walk or bike, etc. Exemplary datastores 106 may include, but are not limited to, a datastore of insurance-related information, a datastore of financial-related information, a datastore of property record information, a datastore of social media information, and a datastore of map and/or traffic information. Information and/or data may be stored in the datastores 106 using any number and/or type(s) of data structures. The datastores 106 may be stored on any number and/or type(s) of non-transitory computer and/or machine-readable medium.

To determine whether a person is likely to perform a wellness activity, the exemplary wellness activity server 102 may include an exemplary propensity model 108. The information and/or data accessed by the data miner 104 may be processed by the propensity model 108 to determine a propensity score that represents a likelihood that the person would perform a wellness activity identified based upon the accessed information and/or data.

In some examples, the propensity model 108 may include a machine learning algorithm 110. The machine learning algorithm 110 may be initially trained using training data representing wellness activities suggested for a plurality of other persons and their feedback, responses, etc. (e.g., ignored, no interest, potential interest, declined, maybe, completed, etc.). An individualized instance of the machine learning model 110 of the propensity model 108 may then be updated, adjusted, trained etc. by an exemplary model updater 112 for each person based on their specific, individual, unique, etc. wellness activity recommendations, feedback, responses, etc. Thus, over time the machine learning algorithm 110 and, more generally, the propensity model 108 become more accurate in identifying when the information and/or data accessed by the data miner 104 may represent a tailored wellness activity that a person will be interested in completing.

To identify wellness activities, the wellness activity server 102 includes an exemplary activity identifier 114. When, for example, the propensity score determined by the propensity model 108 satisfies a condition (e.g., exceeds a predetermined threshold), the activity identifier 114 may query a wellness activity datastore 116 based upon an aspect, keywords, etc. of the information and/or data accessed by the data miner 104. For example, if the information and/or data accessed by the data miner 104 identified a piece of exercise equipment owned by a person (e.g., contained the keyword “bicycle”), the activity identifier 114 may identify in the wellness activity datastore 116 wellness activities involving the piece of exercise equipment. If, for example, the accessed information and/or data identified a bicycle, and a wellness activity identified in the wellness activity datastore 116 includes biking, the activity identifier 114 may query the accessed information and/or data for information related to a home location and a work location.

Additionally and/or alternatively, the data miner 104 may access the datastores 106 for additional information and/or data related to a home location and/or a work location. Based on the additional information and/or data, the activity identifier 114 may modify a generic “biking” wellness activity to a tailored, suggested “biking to work” wellness activity. Additionally and/or alternatively, the data miner 104 may access the datastores 106 for traffic information, and the activity identifier 114 may further modify the “biking to work” wellness activity to a “biking to work via this safer route” wellness activity. In another example, if the information and/or data accessed by the data miner 104 contained an indication of the opening of a new park, the activity identifier 114 may identify a generic “walking” wellness activity in the wellness activity datastore 116, and then modify it to be a tailored, suggested “walking in the new park” wellness activity.

The wellness activity server 102 may communicate, present, convey, etc. information regarding the identified, suggested, tailored wellness activities identified by the activity identifier 114 to a person 117 via, for example, a wellness activity user interface (UI) 118 on an electronic device 120. The wellness activity UI 118 may be, for example, a web-based UI, a dedication application, etc. In some examples, the person 117 can provide feedback, responses, etc. (e.g., ignored, no interest, potential interest, declined, maybe, completed, etc.) to the tailored wellness activities. The electronic device 120 may be any number and/or type(s) of electronic device including, but not limited to, a personal computer, a laptop computer, a mobile device (e.g., a cell phone, a smart phone, a tablet, or a smart watch), a personal digital assistant (PDA), a gaming console, a headset, watch or other wearable device, and/or any other type of computing device.

The wellness activity server 102 may communicate the wellness activities via any number and/or type(s) of network(s) 122 including, but not limited to, a wireless local area network (WLAN), a wireless hotspot, a cellular network, an Ethernet network, an asynchronous transfer mode (ATM) network, a digital subscriber line (DSL) connection, a dialup connection, a satellite network, a coaxial cable network, etc.

In some examples, the electronic device 120 may include a monitoring application 124 for automatically monitoring, tracking, measuring, etc. information and/or data related to the completion wellness activities. The monitoring application 124 may monitor information such as, but not limited to, steps walked, heart rate, routes taken or places visited by the person 117, etc.

The wellness activity server 102 may include a monitor system 126 for monitoring for feedback on, responses to, completion of, etc. presented wellness activities. The monitor system 126 may receive feedback, responses, etc. entered by the person 117 via the wellness activity UI 118 and/or automatically collected by the monitoring application 124. The monitor system 126 may provide the collected feedback, responses, completion information, etc. to the model updater 112 for use in, for example, updating the machine learning algorithm 110 or, more generally, the propensity model 108.

For completed wellness activities, the monitor system 126 may notify an incentive system 128 so the person 117 may be awarded incentives associated with completing wellness activities. For example, the person 117 may be awarded a coupon, a discount, etc. for wellness or health related services or products. In some examples, incentives may be indicated together with suggested wellness activities.

While an exemplary manner of implementing the wellness activity server 102 is illustrated in FIG. 1, one or more of the elements, processes, systems, devices, etc. illustrated in FIG. 1 may be combined, divided, re-arranged, omitted, eliminated and/or implemented in any other way. Further, the data miner 104, the propensity model 108, the machine learning algorithm 110, the model updater 112, the activity identifier 114, the monitor system 126, the incentive system 128 and/or, more generally, the wellness activity server 102 of FIG. 1 may be implemented by hardware, software, firmware and/or any combination of hardware, software and/or firmware. Thus, for example, any of the data miner 104, the propensity model 108, the machine learning algorithm 110, the model updater 112, the activity identifier 114, the monitor system 126, the incentive system 128 and/or, more generally, the wellness activity server 102 could be implemented by one or more of an analog circuit, a digital circuit, a logic circuit, a programmable processor, a programmable controller, a graphics processing unit (GPU), a digital signal processor (DSP), an application specific integrated circuit (ASIC), a programmable logic device (PLD), a field programmable gate array (FPGA), and/or a field programmable logic device (FPLD). Further still, the wellness activity server 102 of FIG. 1 may include one or more elements, processes, systems, devices, etc. in addition to, or instead of, those illustrated in FIG. 1, and/or may include more than one of any or all of the illustrated elements, processes, systems, devices, etc.

### Exemplary Flowcharts

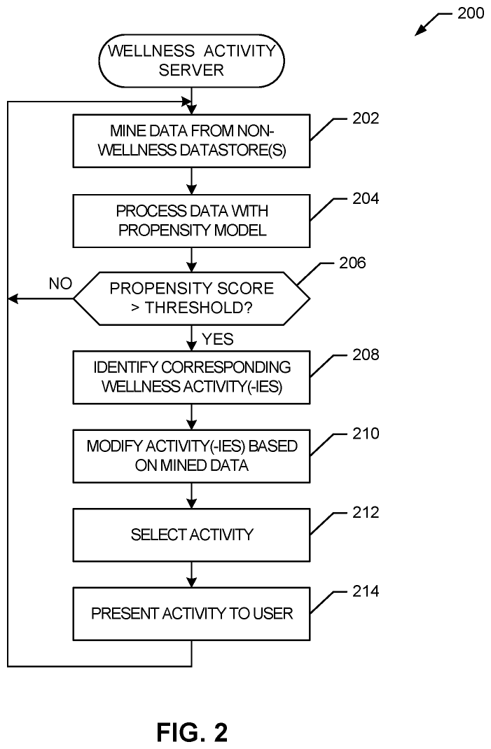

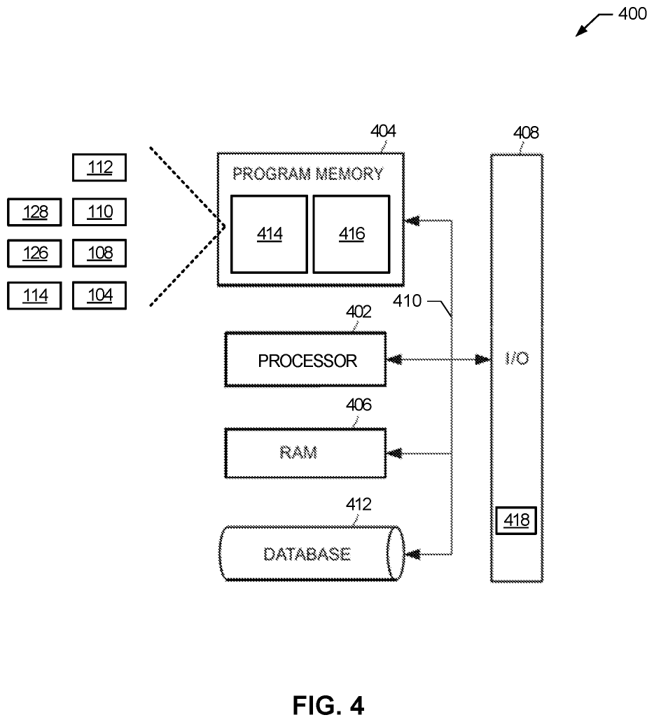

FIG. 2 illustrates a flowchart 200 representative of exemplary processes, methods, software, computer- or machine-readable instructions, etc. for implementing the wellness activity server 102 of FIG. 1. The processes, methods, software and instructions may be an executable program or portion of an executable program for execution by a processor such as the processor 402 shown in an exemplary computing system 400 discussed below in connection with FIG. 4. The program may be embodied in software or instructions stored on a non-transitory computer- or machine-readable storage medium such as a compact disc (CD), a hard drive, a digital versatile disk (DVD), a Blu-ray disk, a cache, a flash memory, a read-only memory (ROM), a random access memory (RAM), or any other storage device or storage disk associated with the processor 402 in which information is stored for any duration (e.g., for extended time periods, permanently, for brief instances, for temporarily buffering, and/or for caching of the information). Further, although the exemplary program is described with reference to the flowchart 200 illustrated in FIG. 2, many other methods of implementing the wellness activity server 102 may alternatively be used. For example, the order of execution of the blocks may be changed, and/or some of the blocks described may be changed, eliminated, or combined. Additionally, or alternatively, any or all of the blocks may be implemented by one or more hardware circuits (e.g., discrete and/or integrated analog and/or digital circuitry, an ASIC, a PLD, an FPGA, an FPLD, a logic circuit, etc.) structured to perform the corresponding operation without executing software or instructions.

The exemplary flowchart 200 begins with the wellness activity server 102 (e.g., the data miner 104) accessing one or more non-wellness related datastores (e.g., the datastores 106) to obtain, collect, access, etc. information and/or data from which wellness activities may potentially be identified (block 202). The wellness activity server 102 (e.g., the propensity model 108) may process the accessed information and/or data with, for example, a machine learning algorithm 110, to determine a propensity score that represents a likelihood that a person may perform a wellness activity identified based upon the accessed information and/or data (block 204).

When the propensity score satisfies a condition (e.g., exceeds a predetermined threshold) (block 206), then the wellness activity server 102 (e.g., the activity identifier 114) may query a wellness activity datastore (e.g., the wellness activity datastore 116) based upon an aspect, keyword, etc. of the accessed information and/or data to identify wellness activities (block 208). The activity identifier 114 may modify any identified wellness activities based upon other aspects of the accessed information and/or data, and/or additional non-wellness-related information and/or data accessed by the data miner 104 (block 210).

The activity identifier 114 may select one or more thus identified and/or modified tailored wellness activities (block 212), and present the selected tailored wellness activity(-ies) via, for example a user interface of a person's electronic device (block 214). Control then returns to block 202 to mine for additional, applicable non-wellness-related information and/or data.

Returning to block 206, if the propensity score does not satisfy the condition (block 206), control returns to block 202 to mine for additional, applicable non-wellness-related information and/or data.

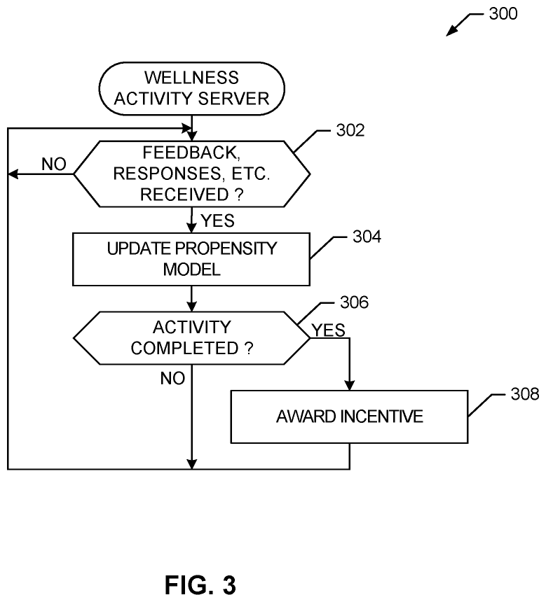

FIG. 3 illustrates a flowchart 300 representative of exemplary hardware logic, machine-readable instructions, hardware-implemented state machines, and/or any combination thereof for implementing the wellness activity server 102 of FIG. 1. The machine-readable instructions may be an executable program or portion of an executable program for execution by a computer processor such as the processor 402 shown in the exemplary computing system 400 discussed below in connection with FIG. 4.

The program may be embodied in software stored on a non-transitory computer-readable storage medium such as a CD, a CD-ROM, a floppy disk, a hard drive, a DVD, a Blu-ray disk, or a memory associated with the processor 402, but the entire program and/or parts thereof could alternatively be executed by a device other than the processor 402 and/or embodied in firmware or dedicated hardware. Further, although the exemplary program is described with reference to the flowchart 300 illustrated in FIG. 3, many other methods of implementing the wellness activity server 102 may alternatively be used. For example, the order of execution of the blocks may be changed, and/or some of the blocks described may be changed, eliminated, or combined. Additionally, and/or alternatively, any or all of the blocks may be implemented by one or more hardware circuits (e.g., discrete and/or integrated analog and/or digital circuitry, an FPGA, an ASIC, a PLD, an FPLD, a comparator, an operational-amplifier (op-amp), a logic circuit, etc.) structured to perform the corresponding operation without executing software or firmware.

The exemplary flowchart 300 of FIG. 3 begins with the wellness activity server 102 (e.g., the monitor system 126) waiting to receive feedback, responses, completion information, etc. regarding suggested, tailored wellness activities (block 302). When feedback, responses, completion information, etc. are received (block 302), the monitor system 126 may provide the feedback, responses, completion information, etc. to, for example, the model updater 112, which may update a propensity model (e.g., the machine learning algorithm 110 and/or, more generally, the propensity model 108) based upon the feedback, responses, completion information, etc. (block 304).

The wellness activity server 102 (e.g., the monitor system 126) may determine whether the feedback, responses, completion information, etc. indicate a recommended, tailored wellness activity has been completed (block 306). If a wellness activity has been completed (block 306), the monitor system 126 may notify, for example, the incentive system 128 to award an incentive for completing the wellness activity (block 308). Control returns to block 302 to continue monitoring for feedback, responses, etc.

### Exemplary Computing System

Referring now to FIG. 4, a block diagram of an exemplary computing system 400 for recommending tailored wellness activities based upon non-wellness-related data, in accordance with described embodiments. The exemplary computing system 400 may be used to, for example, implement all or part of the data miner 104, the propensity model 108, the machine learning algorithm 110, the model updater 112, the activity identifier 114, the monitor system 126, the incentive system 128 and/or, more generally, the wellness activity server 102 of FIG. 1. The computing system 400 may be, for example, a server, a personal computer, a workstation or any other type of computing device

The computing system 400 includes a processor 402, a program memory 404, a RAM 406, and an input/output (I/O) circuit 408, all of which are interconnected via an address/data bus 410. The program memory 404 may store software, and machine- or computer-readable instructions, which may be executed by the processor 402.

It should be appreciated that although FIG. 4 depicts only one processor 402, the computing system 400 may include multiple processors 402. Moreover, different portions of the exemplary wellness activity server 102 may be implemented by different computing systems such as the computing system 400. The processor 402 of the illustrated example is hardware, and may be a semiconductor based (e.g., silicon based) device. Exemplary processors 402 include a programmable processor, a programmable controller, a GPU, a DSP, an ASIC, a PLD, an FPGA, an FPLD, etc. In this example, the processor 402 may implement the data miner 104, the propensity model 108, the machine learning algorithm 110, the model updater 112, the activity identifier 114, the monitor system 126, and/or the incentive system 128.

The program memory 404 may include volatile and/or non-volatile memories, for example, one or more RAMs (e.g., a RAM 414) or one or more program memories (e.g., a ROM 416), or a cache (not shown) storing one or more corresponding software, and machine- or computer-instructions. For example, the program memory 404 stores software, and machine- or computer-readable instructions, or computer-executable instructions that may be executed by the processor 402 to implement any of the data miner 104, the propensity model 108, the machine learning algorithm 110, the model updater 112, the activity identifier 114, the monitor system 126, the incentive system 128 and/or, more generally, the wellness activity server 102 for recommending tailored wellness activities based upon non-wellness-related data. Modules, systems, etc. instead of and/or in addition to those shown in FIG. 4 may be implemented. The software, machine-readable instructions, or computer-executable instructions may be stored on separate non-transitory computer- or machine-readable storage mediums or disks, or at different physical locations.

Exemplary memories 404, 414, 416 include any number or type(s) of volatile or non-volatile non-transitory computer- or machine-readable storage medium or disk, such as semiconductor memories, magnetically readable memories, optically readable memories, hard disk drive (HDD), an optical storage drive, a solid-state storage device, a solid-state drive (SSD), a read-only memory (ROM), a random-access memory (RAM), a CD, a CD-ROM, a DVD, a Blu-ray disk, a redundant array of independent disks (RAID) system, a cache, a flash memory, or any other storage device or storage disk in which information may be stored for any duration (e.g., permanently, for an extended time period, for a brief instance, for temporarily buffering, for caching of the information, etc.).

As used herein, the term non-transitory computer-readable medium is expressly defined to include any type of computer-readable storage device and/or storage disk and to exclude propagating signals and to exclude transmission media. As used herein, the term non-transitory machine-readable medium is expressly defined to include any type of machine-readable storage device and/or storage disk and to exclude propagating signals and to exclude transmission media.

In some embodiments, the processor 402 may also include, or otherwise be communicatively connected to, a database 412 or other data storage mechanism (one or more hard disk drives, optical storage drives, solid state storage devices, CDs, CD-ROMs, DVDs, Blu-ray disks, etc.). In the illustrated example, the database 412 stores the datastore(s) 106 and/or the datastore 116.

Although FIG. 4 depicts the I/O circuit 408 as a single block, the I/O circuit 408 may include a number of different types of I/O circuits or components that enable the processor 402 to communicate with peripheral I/O devices. Exemplary interface circuits 408 include an Ethernet interface, a universal serial bus (USB), a Bluetooth® interface, a near field communication (NFC) interface, and/or a PCI express interface. The peripheral I/O devices may be any desired type of I/O device such as a keyboard, a display (a liquid crystal display (LCD), a cathode ray tube (CRT) display, a light emitting diode (LED) display, an organic light emitting diode (OLED) display, an in-place switching (IPS) display, a touch screen, etc.), a navigation device (a mouse, a trackball, a capacitive touch pad, a joystick, etc.), a speaker, a microphone, a printer, a button, a communication interface, an antenna, etc.

The I/O circuit 408 may include a number of different network transceivers 418 that enable the computing system 400 to communicate with another computer system, such as the electronic device 120, to convey recommended, tailored wellness activities via, for example, a network (e.g., the communication network(s) 122). The network transceiver 418 may be a wireless fidelity (Wi-Fi) transceiver, a Bluetooth transceiver, an infrared transceiver, a cellular transceiver, an Ethernet network transceiver, an ATM network transceiver, a DSL modem, a dialup modem, a satellite transceiver, a coaxial cable modem, etc.

Use of “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise. A device or structure that is “configured” in a certain way is configured in at least that way, but may also be configured in ways that are not listed.

Further, as used herein, the expressions “in communication,” “coupled” and “connected,” including variations thereof, encompasses direct communication and/or indirect communication through one or more intermediary components, and does not require direct mechanical or physical (e.g., wired) communication and/or constant communication, but rather additionally includes selective communication at periodic intervals, scheduled intervals, aperiodic intervals, and/or one-time events. The embodiments are not limited in this context.

Further still, unless expressly stated to the contrary, “or” refers to an inclusive or and not to an exclusive or. For example, “A, B or C” refers to any combination or subset of A, B, C such as (1) A alone, (2) B alone, (3) C alone, (4) A with B, (5) A with C, (6) B with C, and (7) A with B and with C. As used herein, the phrase “at least one of A and B” is intended to refer to any combination or subset of A and B such as (1) at least one A, (2) at least one B, and (3) at least one A and at least one B. Similarly, the phrase “at least one of A or B” is intended to refer to any combination or subset of A and B such as (1) at least one A, (2) at least one B, and (3) at least one A and at least one B.

Moreover, in the foregoing specification, specific embodiments have been described. However, one of ordinary skill in the art appreciates that various modifications and changes can be made in view of aspects of this disclosure without departing from the scope of the invention as set forth in the claims below. Accordingly, the specification and figures are to be regarded in an illustrative rather than a restrictive sense, and all such modifications made in view of aspects of this disclosure are intended to be included within the scope of present teachings.

Additionally, the benefits, advantages, solutions to problems, and any element(s) that may cause any benefit, advantage, or solution to occur or become more pronounced are not to be construed as a critical, required, or essential features or elements of any or all the claims.

Furthermore, although certain exemplary methods, apparatus and articles of manufacture have been disclosed herein, the scope of coverage of this patent is not limited thereto. On the contrary, this patent covers all methods, apparatus and articles of manufacture fairly falling within the scope of the claims of this patent.

Finally, any references, including, but not limited to, publications, patent applications, and patents cited herein are hereby incorporated in their entirety by reference to the same extent as if each reference were individually and specifically indicated to be incorporated by reference and were set forth in its entirety herein.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

Although certain exemplary methods, apparatus and articles of manufacture have been disclosed herein, the scope of coverage of this patent is not limited thereto. On the contrary, this patent covers all methods, apparatus and articles of manufacture fairly falling within the scope of the claims of this patent.

What is claimed is:

1. A computer-implemented method for recommending wellness activities based upon non-wellness-related data, the method comprising:
accessing non-wellness-related data for a person from a datastore;
processing, using one or more processors, the data with a propensity model to determine a propensity score, wherein the propensity score represents a likelihood that the person would perform a wellness activity;
when the propensity score satisfies a condition, identifying, using one or more processors, a wellness activity related to an aspect of the data; and
communicating, via a network interface, information regarding the wellness activity to the person.

2. The computer-implemented method of claim 1, further comprising updating the propensity model based upon feedback regarding the wellness activity.

3. The computer-implemented method of claim 2, wherein the feedback includes an indication from the person of at least one of no interest in the wellness activity, potential interest in the wellness activity, or completion of the wellness activity.

4. The computer-implemented method of claim 2, wherein the feedback is generated by a personal computing device that automatically tracks completion of wellness activities.

5. The computer-implemented method of claim 1, further comprising:
collecting, using one or more processors, feedback regarding the wellness activity; and
awarding, using one or more processors, an incentive based upon the feedback.

6. The computer-implemented method of claim 1, wherein the propensity model includes a machine learning algorithm updated for the person based upon feedback regarding the wellness activity.

7. The computer-implemented method of claim 1, further comprising modifying an aspect of the wellness activity based upon additional non-wellness-related data for the person from the datastore or another datastore.

8. The computer-implemented method of claim 1, wherein the datastore stores at least one of insurance-related information, financial-related information, property record information, or social media information.

9. The computer-implemented method of claim 1, wherein the data represents ownership of a piece of equipment, and the identified wellness activity includes a use of the piece of equipment.

10. The computer-implemented method of claim 1, wherein the data represents opening of a new wellness activity area, and the wellness activity includes use of the new wellness activity area.

11. A computer system for recommending wellness activities based upon non-wellness-related data, the system comprising:
a data miner configured to access non-wellness-related data for a person from a datastore;
a propensity model configured to process the data to determine a propensity score, wherein the propensity score represents a likelihood that the person would perform a wellness activity;
an activity identifier configured to, when the propensity score satisfies a condition, identify a wellness activity related to an aspect of the data; and
a network interface configured to communicate information regarding the wellness activity to the person.

12. The system of claim 11, further comprising a monitor system configured to collect feedback regarding the wellness activity, wherein the propensity model is configured to update based upon the feedback.

13. The system of claim 11, further comprising:
a monitor system configured to collect feedback regarding the wellness activity; and
an incentive system configured to award an incentive based upon the feedback.

14. The system of claim 11, wherein the activity identifier is configured to modify an aspect of the wellness activity based upon additional non-wellness-related data for the person from the datastore or another datastore.

15. The system of claim 11, wherein the datastore stores at least one of insurance-related information, financial-related information, property record information or social media information.

16. A non-transitory computer-readable storage medium comprising instructions that, when executed by one or more processors, cause a system to:
access non-wellness-related data for a person from a datastore;
process the data to determine a propensity score, wherein the propensity score represents a likelihood that the person would perform a wellness activity;
when the propensity score satisfies a condition, identify a wellness activity related to an aspect of the data; and
communicate information regarding the wellness activity to the person.

17. The non-transitory computer-readable storage medium of claim 16, wherein the instructions, when executed by the one or more processors, cause the system to:
collect feedback regarding the wellness activity; and
update a model used to process the propensity score based upon the feedback.

18. The non-transitory computer-readable storage medium of claim 16, wherein the instructions, when executed by the one or more processors, cause the system to:
collect feedback regarding the wellness activity; and
award an incentive based upon the feedback.

19. The non-transitory computer-readable storage medium of claim 16, wherein the instructions, when executed by the one or more processors, cause the system to modify an aspect of the wellness activity based upon additional non-wellness-related data for the person from the datastore or another datastore.

20. The non-transitory computer-readable storage medium of claim 16, wherein the datastore stores at least one of insurance-related information, financial-related information, property record information or social media information.
